(19) United States
(12) Patent Application Publication
(10) Pub. No.: US 2025/0217720 A1
(43) Pub. Date: Jul. 3, 2025

(54) FUSIONNET AND ENSEMBLE LEARNING-BASED ARTIFICIAL INTELLIGENCE SYSTEM FOR ALZHEIMER'S DISEASE CLASSIFICATION FROM OPTICAL COHERENCE TOMOGRAPHY THICKNESS AND DEVIATION MAPS

(71) Applicant: The Chinese University of Hong Kong, Hong Kong (CN)

(72) Inventors: Yim Lui Carol CHEUNG, Hong Kong (CN); Anran RAN, Hong Kong (CN); Chung Tong Vincent MOK, Hong Kong (CN); Chee Yung Clement THAM, Hong Kong (CN)

(21) Appl. No.: 19/004,686

(22) Filed: Dec. 30, 2024

Related U.S. Application Data
(60) Provisional application No. 63/615,868, filed on Dec. 29, 2023.

(51) Int. Cl. G06N 20/20; G06T 7/00; G06V 10/77; G06V 10/80; G16H 50/20

(52) U.S. Cl. CPC G06N 20/20; G06T 7/0012; G06V 10/7715; G06V 10/806; G16H 50/20; G06T 2207/10101

(57) ABSTRACT

The subject invention pertains to deep learning (DL) systems and methods for the binary classification of Alzheimer's Disease. Optical coherence tomography (OCT) generated reports were used, and the images were grouped into 3 different inputs: (1) an optic nerve head (ONH) model including a retinal nerve fiber layer (RNFL) thickness map, a RNFL deviation map, and an ONH-centered en face image; (2) a Macula model including a ganglion cell inner plexiform layer (GCIPL) thickness map, a GCIPL deviation map, a macular thickness map, and a macula-centered en face image; and (3) a General model including all images of (1) and (2). A fusion network is provided to analyze the plurality of images from a single eye for classification. The fusion network includes Feature Extraction, Feature Fusion, and Feature Reconstruction. Ensemble learning is provided to advantageously combine several baseline models to build a single but more powerful model.

### CROSS-REFERENCE TO RELATED APPLICATION

The present application claims the benefit of U.S. Provisional Application Ser. No. 63/615,868, filed Dec. 29, 2023, the disclosure of which is incorporated by reference herein in its entirety.

### BACKGROUND OF THE INVENTION

Alzheimer's disease (AD) remains as the leading cause of dementia worldwide. While current treatment for AD generally abides by the principle of symptom-relief, the recent approval of two drugs, aducanumab and lecanemab, by the U.S. Food and Drug Administration signifies a paradigm shift in the management of AD, from a sole symptomatic treatment approach to the exploration of disease-modifying therapies. These monoclonal antibodies selectively bind to aggregated forms of amyloid beta and reduce the accumulation of Aβ plaques in the human brain, which was believed to be the culprit of cognitive decline in AD pathogenesis. Although studies regarding the clinical benefit of aducanumab and lecanemab on real-life patients remain inconclusive, their use was generally recommended for individuals with mild cognitive impairment or mild dementia due to AD, which calls for an unprecedented need for the development of a feasible screening tool that is sensitive towards the detection of early AD-associated changes once more convincing evidence arises.

While in-vivo neuroimaging modalities such as MRI and positron emission tomography (PET) achieve high accuracy in AD detection, these modalities are often limited by its high cost, low accessibility, invasiveness, technical complexity, and the risk of using radioactive tracers. Given the status quo, scientists have actively explored other viable markers of AD. With evidence revealing the presence of pathophysiological markers before the manifestation of clinical symptoms, the retina has been considered a window to study AD as an accessible extension of the brain in terms of embryology, anatomy, and physiology.

Optical coherence tomography (OCT) has enabled detailed investigation and quantification of individual retinal layers including retinal neuronal and axonal structures such as retinal nerve fiber layer (RNFL), the ganglion cell layer (GCL), and the inner plexiform layer (IPL) non-invasively. The quantitative segmental analysis of retinal layers enabled scientists to study their relationship with cognitive function impairment. Neuronal loss in the hippocampus and cerebral neocortex is a typical characteristic of AD. The loss of RGCs and their axons in AD patients is usually exhibited in the RNFL surrounding the optic nerve head (peripapillary RNFL). RNFL thinning, especially in the superior and inferior quadrants, is observed in patients with AD in multiple OCT studies. Macular GCIPL thickness can also be measured through OCT, which demonstrates thinning around fovea under the manifestation of AD. While the relative accuracy and sensitivity between the two parameters remain controversial, the association with AD demonstrates potential use as indicators of neurodegeneration for assisting early diagnosis.

Concomitantly, the evolving field of artificial intelligence, particularly deep learning (DL), presents unique opportunities to the detection of AD from retinal photographs (Marshall and Uchegbu, Tian, Smith et al. 2021, Cheung, Ran et al. 2022, Wisely, Wang et al. 2022). Cheung et al. (Cheung, Ran et al. 2022) has recently developed a DL algorithm based on 4 retinal photographs (optic nerve head and macula-centered fields from both eyes) for each subject for detecting AD-dementia, which discriminated Aβ-positive from Aβ-negative with accuracy, sensitivity, and specificity ranging from 80.6% to 89.3%, 75.4% to 90.0%, and 92.0% to 100.0%, respectively, in testing datasets with data on PET. Wisely et al. (Wisely, Wang et al. 2022) proposed a multimodal DL system to predict AD using images and measurements from multiple ocular imaging modalities (OCT, OCT angiography, ultra-widefield retinal photography, and retinal autofluorescence), which achieved the highest area under the receiver operating characteristic curve (AUROC) of 0.861 on the validation set and 0.841 on the test set. Tian et al. (Tian, Smith et al. 2021) has developed a highly modular DL algorithm that enables automated image selection, vessel segmentation, and classification of AD, achieving an accuracy of over 80%.

Alzheimer's disease (AD), the most common form of dementia, is a major public health and clinical challenge globally, causing a significant socioeconomic burden worldwide. With evidence revealing the presence of pathophysiological markers before the manifestation of clinical symptoms, the retina has been considered a window to study AD as an accessible extension of the brain in terms of embryology, anatomy, and physiology. Advancements in retinal imaging modalities, such as optical coherence tomography (OCT) has enabled detailed investigation and quantification of the neuronal structures of the retina non-invasively and the retina demonstrates potential as a viable platform for the study of neurodegenerative diseases.

The role of DL approaches in identifying AD from OCT alone remains under development. Training of more complex deep neural networks can increase the chance of overfitting.

### BRIEF SUMMARY OF THE INVENTION

The evolving field of artificial intelligence, particularly deep learning (DL), presents unique opportunities for the detection of AD from retinal photographs, while the role of DL approaches in identifying the ocular neurodegenerative changes of patients with AD from OCT alone has yet to be determined. The training of more complex deep neural networks increases the chance of overfitting. Thus, ensemble learning is provided in certain embodiments due to its advantage of combining several baseline models to build a single but more powerful model. Embodiments provide a novel FusionNet and ensemble learning-based DL methods for the automated detection of AD dementia using a plurality of different OCT inputs from a single respective eye, including retinal nerve fiber layer (RNFL) thickness map, RNFL deviation map, macula thickness map, ganglion cell inner plexiform layer (GCIPL) thickness map, GCIPL deviation map, optic nerve head (ONH)-centered and macula-centered en face images, with precise AD screening as the ultimate aim. Embodiments provide different combinations of OCT inputs in constructing the provided DL models, which can maximize performance in AD detection as well as providing insight into the underlying mechanism and improving interpretability. The inadequacy in the potential bias is also addressed through ensemble learning, embodiments of which combine multiple DL methods to achieve better predictive performance when processing highly diversified inputs.

Embodiments of the subject invention provide Alzheimer's disease or dementia treatment and/or screening, and/or systems or methods for Alzheimer's disease or dementia treatment and/or screening of a patient including or as a result of a binary classification or other output generated by one or more embodiments disclosed herein, the binary classification or other output identifying the patient as likely having either dementia or no dementia. After the screening, a comprehensive neurological evaluation is needed for final diagnosis and further treatment. The treatment can compose monoclonal antibody or other treatments, for example, to reduce the accumulation of amyloid beta plaques in the human brain. By way of example and not limitation, two such treatments include administration of a therapeutically effective amount of either aducanumab, lecanemab, or a series or combination including one or both.

### BRIEF DESCRIPTION OF THE DRAWINGS

FIG. 1A illustrates the Fusion Network structure comprising (alternatively, consisting of, or consisting essentially of) three components: Feature Extraction, Feature Fusion, and Feature Reconstruction for an ONH model according to an embodiment of the subject invention.

FIG. 1B illustrates the Fusion Network structure comprising (alternatively, consisting of, or consisting essentially of) three components: Feature Extraction, Feature Fusion, and Feature Reconstruction for a macula model according to an embodiment of the subject invention.

FIG. 1C illustrates the Fusion Network structure comprising (alternatively, consisting of, or consisting essentially of) three components: Feature Extraction, Feature Fusion, and Feature Reconstruction for a general model comprising all image sources of FIG. 1A and FIG. 1B.

FIG. 1D illustrates a detailed view, enlarged to show details of the Feature Extraction from the left side of FIG. 1A, for an ONH model according to an embodiment of the subject invention. The enlarged details are similar to and repeated in FIG. 1B and FIG. 1C, while accommodating a multiplicity of different inputs in each respective case.

FIG. 1E illustrates a detailed view, enlarged to show details of the Feature Fusion and Domain Specific Batch Normalization from the central area of FIG. 1A, for an ONH model according to an embodiment of the subject invention. The enlarged details are similar to and repeated in FIG. 1B and FIG. 1C, while accommodating a multiplicity of different inputs in each respective case.

FIG. 1F illustrates a detailed view, enlarged to show details of the Feature Reconstruction and residual blocks from the right side of FIG. 1A, for an ONH model according to an embodiment of the subject invention. The enlarged details are similar to and repeated in FIG. 1B and FIG. 1C, while accommodating a multiplicity of different inputs in each respective case.

FIG. 2 illustrates the structure of the ensemble model according to an embodiment of the subject invention.

### DETAILED DISCLOSURE OF THE INVENTION

In certain embodiments of the subject invention systems and methods comprising ensemble learning are provided, at least in part to advantageously combine several baseline models to build a single but more powerful model. Embodiments provide a novel FusionNet with ensemble learning-based DL methods for the automated detection of AD dementia using a multiplicity of different inputs of single eye data generated from OCT devices, including but not limited to RNFL thickness map, RNFL deviation map, macula thickness map, GCIPL thickness map, GCIPL deviation map, ONH-centered and macula-centered en face images. Embodiments provide different combinations of inputs in constructing certain DL models, which can maximize performance in AD detection as well as provide insight into the underlying mechanism and improve the interpretability of results. Certain inadequacies in potential bias can also be addressed through ensemble learning, which in certain embodiments combines multiple DL methods to achieve better predictive performance when processing various inputs.

AI models for AD classification are based on either retinal photographs only or multi-modalities which need to conduct multiple neurological and ocular imaging modalities. Embodiments of the subject invention are based on OCT alone for AD classification which is simple and non-invasive while providing more relevant information on neurological changes. Certain embodiments comprise FusionNet and ensemble learning which can utilize multiple input and provide a more robust and non-biased prediction. The application of FusionNet in certain embodiments makes use of comprehensive information from one or more multiplicities of different OCT inputs for the analysis of vascular and neurological changes. The application of ensemble learning in certain embodiments increases the feasibility of DL-based retinal imaging for AD in real-world scenarios, where a single method may not be able to handle the discrepancy between individual subjects.

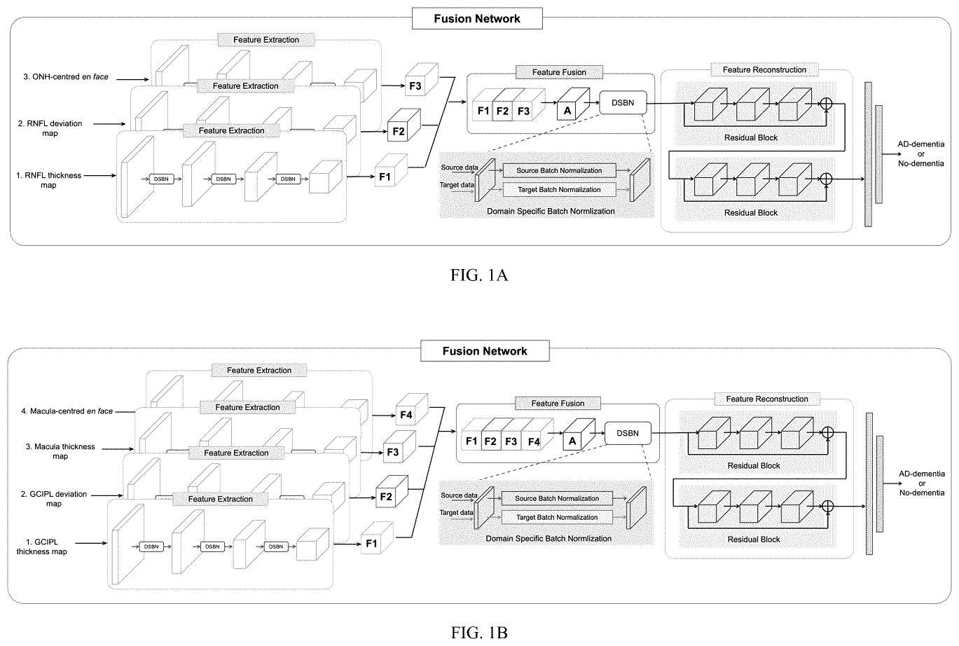

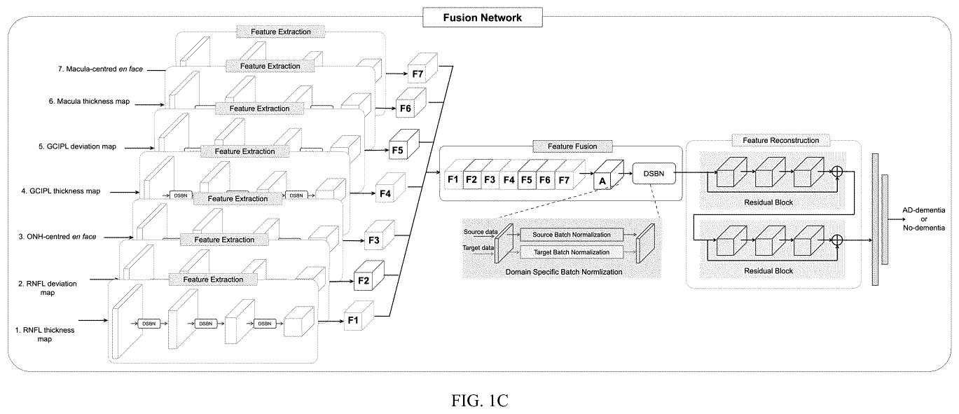

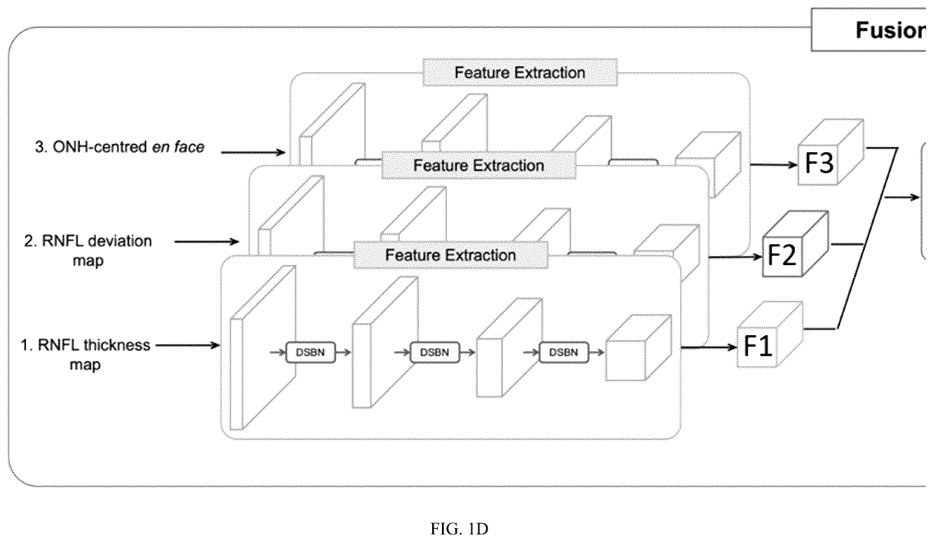

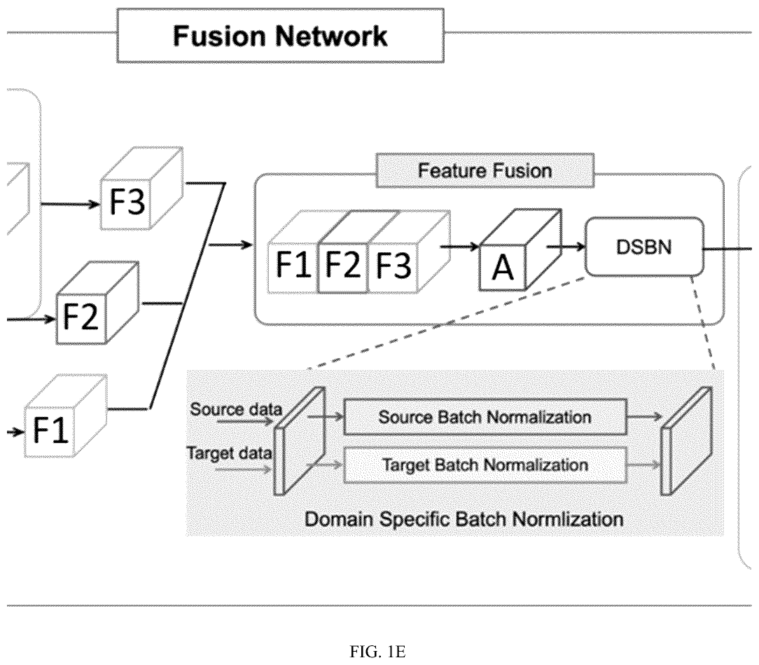

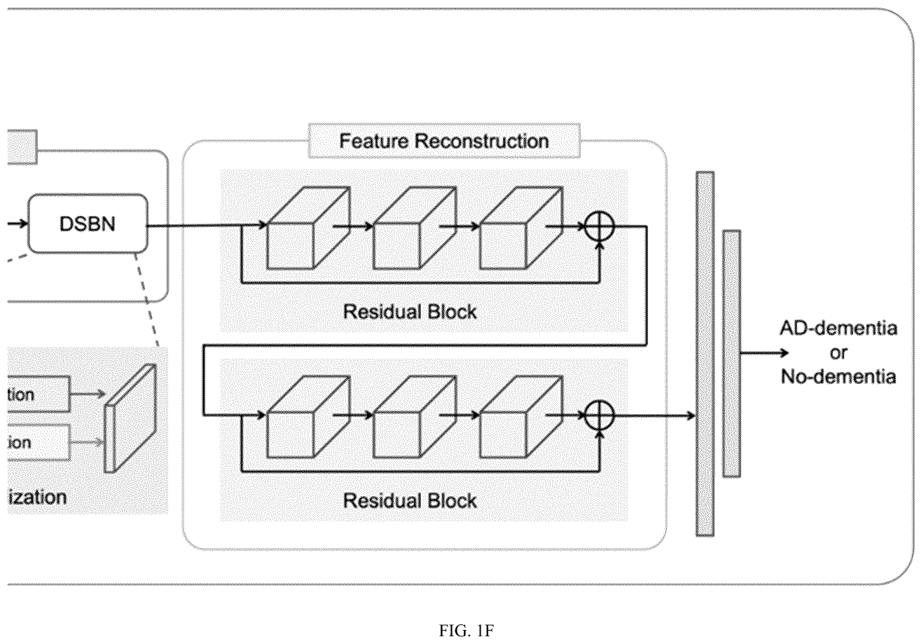

Turning now to the figures, FIG. 1A illustrates the Fusion Network structure comprising (alternatively, consisting of, or consisting essentially of) three components: Feature Extraction, Feature Fusion, and Feature Reconstruction for an ONH model according to an embodiment of the subject invention. In this embodiment, Feature Extraction processes each of 3 respective image input datasets (e.g., 1. RNFL thickness map, 2. RNFL deviation map, and 3. ONH-centered en face images) in parallel, iterating each input through Domain Specific Batch Normalization (DSBN) from an initial input layer to a final output layer (e.g., 4 layers separated by 3 iterations of DSBN in each parallel feature extraction, as illustrated, producing outputs F1, F2, and F3.) Feature Fusion merges outputs F1, F2, and F3 as aggregated features A before passing them through a DSBN layer wherein source data is processed through Source Batch Normalization in parallel with target data processed through Target Batch Normalization to produce a common feature layer. In Feature Reconstruction, the common feature layer is processed through multiple residual blocks and summing junctions (e.g., two iterations in series, each iteration respectively consisting of three residual blocks with a bypass summing junction as shown) to provide a layer as input to a local classifier.

FIG. 1B illustrates the Fusion Network structure comprising (alternatively, consisting of, or consisting essentially of) three components: Feature Extraction, Feature Fusion, and Feature Reconstruction for a macula model according to an embodiment of the subject invention. In this embodiment, Feature Extraction processes each of 4 respective image input datasets (e.g., 1. GCIPL thickness map, 2. GCIPL deviation map, 3. macula thickness map, and 4. macula-centered en face images) in parallel, iterating each input through DSBN from an initial input layer to a final output layer (e.g., 4 layers separated by 3 iterations of DSBN in each parallel feature extraction, as illustrated, producing outputs F1, F2, F3, and F4.) Feature Fusion merges outputs F1, F2, F3, and F4 as aggregated features A before passing them through a DSBN layer wherein source data is processed through Source Batch Normalization in parallel with target data processed through Target Batch Normalization to produce a common feature layer. In Feature Reconstruction, the common feature layer is processed through multiple residual blocks and summing junctions (e.g., two iterations in series, each iteration respectively comprising three residual blocks with a bypass summing junction as shown) to provide a layer as input to a local classifier.

FIG. 1C illustrates the Fusion Network structure comprising (alternatively, consisting of, or consisting essentially of) three components: Feature Extraction, Feature Fusion, and Feature Reconstruction for a general model comprising all image sources of FIG. 1A and FIG. 1B. In this embodiment, Feature Extraction processes each of 7 respective image input datasets, each dataset comprising single eye data, (e.g., 1. RNFL thickness map, 2. RNFL deviation map, 3. ONH-centered en face images, 4. GCIPL thickness map, 5. GCIPL deviation map, 6. macula thickness map, and 7. macula-centered en face images) in parallel, iterating each input through DSBN from an initial input layer to a final output layer (e.g., 4 layers separated by 3 iterations of DSBN in each parallel feature extraction, as illustrated, producing outputs F1 through F7.) Feature Fusion merges outputs F1 through F7 as aggregated features A before passing them through a DSBN layer wherein source data is processed through Source Batch Normalization in parallel with target data processed through Target Batch Normalization to produce a common feature layer. In Feature Reconstruction, the common feature layer is processed through multiple residual blocks and summing junctions (e.g., two iterations in series, each iteration respectively comprising three residual blocks with a bypass summing junction as shown) to provide a layer as an input to a local classifier.

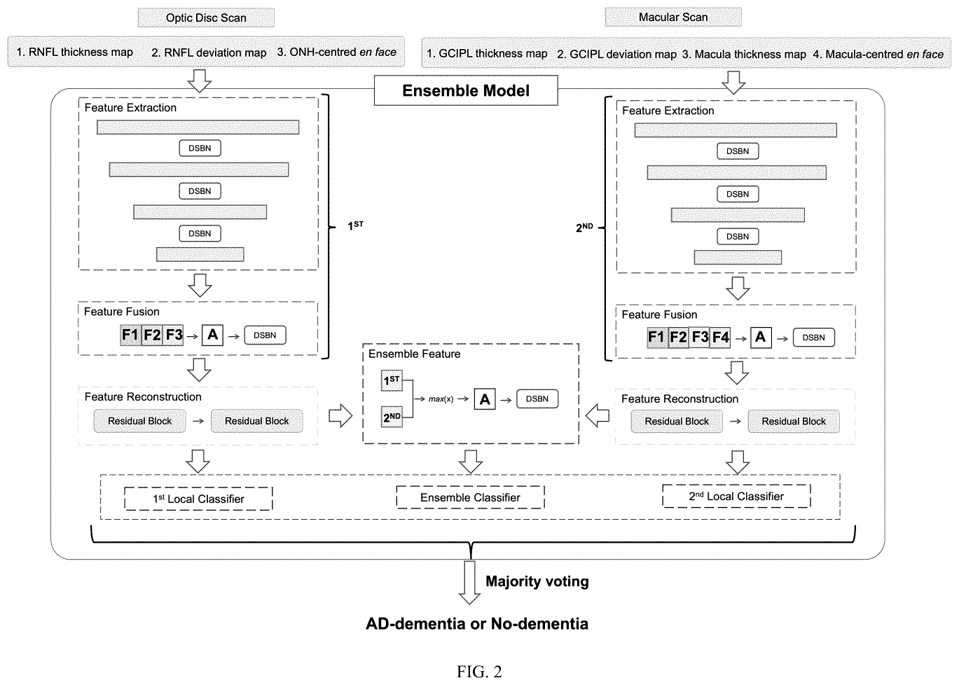

FIG. 2 illustrates the structure of the ensemble model according to an embodiment of the subject invention. This embodiment integrates two fusion networks, the ONH model (e.g., as illustrated in FIG. 1A) and the macula model (e.g., as illustrated in FIG. 1B), to provide a single and unified classification. In addition, the embodiment provides an ensemble feature layer between these two networks to select representative features for the classification. The ensemble feature layer is the summation of multiple features at the channel level. After reconstructed features are generated from these two networks, the ensemble feature layer aggregates and transmits them to the ensemble classifier as well as their respective (e.g., 1^{st} local and 2^{nd} local) classifiers. In the last step, the results of all (e.g., 1^{st} local, ensemble, and 2^{nd} local) classifiers are used to generate the final binary classification results through majority voting.

### Materials and Methods

All patents, patent applications, provisional applications, and publications referred to or cited herein are incorporated by reference in their entirety, including all figures and tables, to the extent they are not inconsistent with the explicit teachings of this specification.

The following are examples that illustrate procedures for practicing the invention. These examples should not be construed as limiting. All percentages are by weight and all solvent mixture proportions are by volume unless otherwise noted.

### Example 1—A Retrospective, Multicenter, Case-Control Approach

The DL methods are trained, validated, and tested for the binary classification of Alzheimer's disease (e.g., classification as AD-dementia or no dementia) using different inputs of a single eye generated from an OCT device according to an embodiment of the subject invention. Specifically, three OCT conventional generated reports were used, namely (1) RNFL, (2) macular thickness (MT), and (3) GCIPL. The GCIPL and RNFL reports contain thickness map and deviation map, while the MT report only contains the thickness map.

Image preprocessing proceeded as follows. The input for development of DL methods comprise thickness maps and deviation maps extracted from the three reports, as well as ONH-centered and macula-centered en face images. Since the variety of images could provide different key information, the images were paired on a patient-level and combined as different inputs. For the model development, the images were grouped into 3 different inputs as follows: (1, as illustrated in FIG. 1A) the ONH model comprising a RNFL thickness map, a RNFL deviation map, and an ONH-centered en face image; (2, as illustrated in FIG. 1B) a Macula model comprising a GCIPL thickness map, a GCIPL deviation map, a macula thickness map, and an macula-centered en face image; (3, as illustrated in FIG. 1C) a General model comprising all images of (1) and (2). The OCT images are pre-processed with data normalization and data augmentation for extracted OCT thickness maps, deviation maps, and en face images. The loop-up table function is additionally applied to enhance the luminance and contrast of original en face images. Each image was resized to the same size, i.e., 450×450 pixels, to ensure efficient feature extraction while keeping the model complexity relatively low.

A fusion network is developed to analyze multiple images from a single eye for AD classification. The fusion network comprises three main components: 1) Feature Extraction, 2) Feature Fusion, and 3) Feature Reconstruction. The model structure is illustrated in FIGS. 1A-1C with a multiplicity of different input images. The Feature Extraction module is built with a structure similar to the visual geometry group network (VGG16) (Simonyan and Zisserman 2015) but only containing its first eight convolutional layers and three max pooling layers. Rectified linear unit (ReLU) activation function and DSBN were employed after convolutional layers. (Nair and Hinton 2010) Each image from the multi-image input was subject to feature extraction and the weights of the convolution kernels were not shared among each feature map from the image, providing one or more multiplicities of feature maps, each respectively having a unique (or uniquely derived) convolution kernel weighting. The Feature Fusion module applied 3D convolution with output channel as 1 and kernel size and stride as (1, 1, 1) over an input signal composed of all the extracted image features to fuse the features together, followed by dimensionality reduction. This provided one or more intermediate nodes, each of which respectively, integrates extracted image features from one or more multiplicities of feature extraction channels. The Feature Reconstruction was then performed on the fused features for new feature representations by extracting deep key information. The procedure involved the 3^{rd} and 4^{th} stage of ResNet50 (He, Zhang et al. 2015) and an additional transition layer utilizing a 2D transposed convolution operator. After obtaining the reconstructed features, 2 fully connected layers were applied to produce a probability score for classification. The loss function of the fusion network was the cross-entropy loss.

Model development and domain adaptation proceeded as follows. The data ratio of AD-dementia patients in the training and validation sets was 9:1 while the subject ratio of no dementia and AD-dementia was 20:1, which took into account the sample balance of the validation set. Individual fusion models were trained based on the different inputs. Each fusion model was trained for 100 epochs using a batch size of 8 and Adam optimization with start learning rate 1e⁻⁴ which decayed every 10 epochs.

The multiplicity of inputs from different datasets includes variances due to factors such as ethnicity and ocular pathologies, which could lead to notable disparity in the DL model performance when testing on different datasets. The domain adaptation approach is applied to address the issue of dataset discrepancies and further enhance the generalizability. Specifically, the training dataset was defined as the source domain, and the external test dataset was defined as the target domain. The model is first trained by supervised learning with labels on the source domain. Subsequently, pseudo-labelling of the target domain was performed with the pre-trained model. In the fusion network, the convolutional layer extracted shared parameters for features from different domains for inter-domain correlation feature learning. The batch normalization layer was established as two branches that accept feature extraction from the source and target domains independently by using domain-specific batch normalization. In certain embodiments the provided domain adaptation can minimize the gap between the two domains in the feature space and achieve the effect that the performance of the depth model in the target domain approximates or even remains in the original domain.

Ensemble learning was further incorporated to integrate all inputs into a single model and achieve optimal performance (e.g., as illustrated in FIG. 2). The multiple fusion networks are combined to design an ensemble model targeting a multiplicity of inputs to provide a single and unified classification. In certain embodiments the multiplicity of inputs comprises a multiplicity of different or unique inputs. In certain embodiments the multiplicity of inputs comprises a number of unique inputs and one or more repeated or overlapping inputs. In this combination scheme, two fusion networks, namely the ONH model and the macula model are integrated. In addition, an ensemble feature layer is constructed between these two networks to select representative features for the classification. The ensemble feature layer is the summation of multiple features at the channel level. After reconstructed features were generated from these two networks, the ensemble feature layer aggregated and transmitted them to the ensemble classifier as well as their respective classifiers. Finally, the results of all classifiers were used to generate the final classification results through majority voting.

The ensemble model is trained with pre-trained parameters of these two fusion networks and froze all layers to optimize only the ensemble feature layer and the classifier. The training process also used the Adam optimization technique and ran through 50 epochs with a batch size of 4 and a learning rate of 1e⁻⁵ initially with decaying every 10 epochs.

Comparison with related art proceeded as follows. Existing AI models for AD classification are based on either FP only or multi-modalities which need to conduct multiple neurological and ocular imaging modalities. Embodiment of the subject invention are advantageously based on OCT alone for AD classification which is simple and non-invasive with more relevant information on neurological changes. In certain embodiments the intervention is based on FusionNet and ensemble learning which can utilize multiple input and provide a more robust and non-bias prediction.

The embodiments of Fusion networks have been tested with a multiplicity of different input images in retrospective multi-center cohorts. The data distribution and the model performance are shown in Table 1 and Table 2, respectively. Embodiments can incorporate a web-based user interface or other advantageously effective access method. Embodiments can comprise an integrated OCT device or OCT scanning step or service.

Selected exemplary and non-limited embodiments of the subject invention include the following.

Embodiment 1 provides pre-processed inputs with data normalization and data augmentation for extracted OCT thickness maps, deviation maps, and en face images. This embodiment applies the loop-up table function to enhance the luminance and contrast of original en face images. Each image can be resized to the same size (e.g., 450×450 pixels) to ensure efficient feature extraction while keeping the model complexity relatively low.

Embodiment 2 provides a fusion network to analyze multiple images from a single eye for AD classification. The fusion network can comprise three components: 1) Feature Extraction, 2) Feature Fusion, and 3) Feature Reconstruction. Each image from the multi-image input can be subjected to feature extraction and the weights of the convolution kernels can be not shared among each feature map from the image. The Feature Fusion provides an intermediate node that integrates all the extracted image features. The Feature Reconstruction can be performed on the fused features for new feature representations by extracting deep key information.

Embodiment 3 incorporated ensemble learning to integrate all inputs into a single model and achieved enhanced performance. This embodiment combined multiple fusion networks to design an ensemble model targeting a multiplicity of different inputs to provide a single and unified classification. In this combination scheme, two fusion networks were integrated, namely the ONH model and the macula model. In addition, an ensemble feature layer was constructed between these two networks to select representative features for the classification. The ensemble feature layer comprised the summation of multiple features at the channel level. After reconstructed features were generated from these two networks, the ensemble feature layer aggregated and transmitted them to the ensemble classifier as well as their respective classifiers. Finally, the results of all classifiers were used to generate the final classification results through majority voting.

Embodiment 4 applied the domain adaptation approach to address the issue of dataset discrepancies and further enhance the generalizability. The model was first trained by supervised learning with labels on the source domain. Subsequently, pseudo-labelling of the target domain was performed with the pre-trained model. In the fusion network, the convolutional layer extracted features shared parameters for features from different domains for inter-domain correlation feature learning. The batch normalization layer was established as two branches that accept feature extraction from the source and target domains independently by using domain-specific batch normalization. The technique of domain adaptation can minimize the gap between the two domains in the feature space and achieve the effect that the performance of the depth model in the target domain approximates or even remains in the original domain.

Embodiment 5 was based on OCT alone for AD classification which is simple and non-invasive with relevantly more information on neurological changes with the provided FusionNet and ensemble learning which can utilize multiple inputs and provide a more robust and non-biased prediction.

Embodiment 6. An ensemble model targeting a multiplicity of inputs to provide a single unified classification, the model comprising:
a first fusion network trained on an optic nerve head (ONH) image dataset; and
a second fusion network trained on a macular image dataset; and
an ensemble feature layer comprising a channel level summation of a multiplicity of features extracted from the first fusion network and a multiplicity of features extracted from the second fusion network.

Embodiment 7. The model according to Embodiment 6, wherein:
the first fusion network comprises a first local classifier;
the second fusion network comprises a second local classifier; and
the ensemble model comprises an ensemble classifier.

Embodiment 8. The model according to Embodiment 7, wherein the first local classifier comprises a first multiplicity of features from the first fusion network; and the second local classifier comprises a second multiplicity of features from the second fusion network.

Embodiment 9. The model according to Embodiment 8, wherein the ensemble classifier comprises a third multiplicity of aggregated features comprising at least one feature from the first multiplicity of features and at least one feature from the second multiplicity of features.

Embodiment 10. The model according to Embodiment 9, wherein the ensemble classifier is configured to return a binary classification indicating either AD-dementia or No-dementia through a majority voting method taking at least one input, respectively, from each of the first multiplicity of features, the second multiplicity of features, and the third multiplicity of aggregated features.

Embodiment 11. The model according to Embodiment 6, wherein:
the first fusion network comprises a first feature extraction component, a first feature fusion component, and a first feature reconstruction component; and
the second fusion network comprises a second feature extraction component, a second feature fusion component, and a second feature reconstruction component.

Embodiment 12. The model according to Embodiment 11, wherein:
the first feature extraction component comprises a first multiplicity of feature maps, each respective feature map in the first multiplicity of feature maps having a unique convolution kernel weighting; and
the second feature extraction component comprises a second multiplicity of feature maps, each respective feature map in the second multiplicity of feature maps having a unique convolution kernel weighting.

Embodiment 13. The model according to Embodiment 11, wherein:
the first feature fusion component comprises a first intermediate node that integrates extracted image features from a first multiplicity of feature extraction channels; and
the second feature fusion component comprises a second intermediate node that integrates extracted image features from a second multiplicity of feature extraction channels.

Embodiment 14. The model according to Embodiment 11, wherein:
the first feature extraction component comprises a first multiplicity of residual blocks; and
the second feature extraction component comprises a second multiplicity of residual blocks.

Embodiment 15. The model according to Embodiment 11, wherein:
the first fusion network comprises a first inter-domain correlation between a first source domain comprising a first training data set and a first target domain comprising a first external test data set; and
the second fusion network comprises a second inter-domain correlation between a second source domain comprising a second training data set and a second target domain comprising a second external test data set.

Embodiment 16. The model according to Embodiment 15, wherein the first fusion network, the second fusion network, and the ensemble feature layer each, respectively, comprises a respective domain specific batch normalization.

Embodiment 17. The model according to Embodiment 16, wherein each respective domain specific batch normalization comprises a first branch configured to accept feature extraction from a respective source domain, and a second branch configured to accept feature extraction from a respective target domain.

Embodiment 18. A method for generating a binary classification of Alzheimer's Disease (AD) in a patient as either (a) AD-dementia or (b) no dementia, the method comprising:
creating a deep learning system by performing the following steps:
   a) providing a first fusion network comprising a first feature extraction module, a first feature fusion module, a first feature reconstruction module, and a first classifier;
   b) training the first fusion network on a first image set comprising a plurality of optical coherence tomography (OCT) optic nerve head (ONH) image channels;
   c) providing a second fusion network comprising a second feature extraction module, a second feature fusion module, a second feature reconstruction module, and a second classifier;
   d) training the second fusion network on a second image set comprising a plurality of optical coherence tomography (OCT) macula-centered image channels;
   e) providing an ensemble model comprising the first fusion network, the second fusion network, an ensemble feature layer connected to an output of the first fusion network and to an output of the second fusion network, and an ensemble classifier;
   f) training the ensemble model on the combined first image set and second image set;
obtaining a patient-specific OCT image set; and
processing the patient-specific OCT image set through the ensemble model, thereby generating the binary classification of Alzheimer's disease in the patient as either (a) AD-dementia or (b) no dementia.

Embodiment 19. The method according to Embodiment 18, wherein the plurality of OCT ONH image channels comprises at least one of retinal nerve fiber layer (RNFL) thickness map images, RNFL deviation map images, and ONH-centered en face images, each respective image channel comprising single eye data.

Embodiment 20. The method according to Embodiment 18, wherein the plurality of OCT ONH image channels comprise retinal nerve fiber layer (RNFL) thickness map images, RNFL deviation map images, and ONH-centered en face images, each respective image channel comprising single eye data.

Embodiment 21. The method according to Embodiment 18, wherein the plurality of OCT macula-centered image channels comprises at least one of ganglion cell inner plexiform layer (GCIPL) thickness map images, GCIPL deviation map images, macular thickness map images, and macula-centered en face images, each respective image channel comprising single eye data.

Embodiment 22. The method according to Embodiment 18, wherein the plurality of OCT ONH image channels comprise ganglion cell inner plexiform layer (GCIPL) thickness map images, GCIPL deviation map images, macular thickness map images, and macula-centered en face images, each respective image channel comprising single eye data.

Embodiment 23. The method according to Embodiment 18, wherein the provided ensemble model is configured to generate the final classification results through majority voting including outputs from the first classifier, the second classifier, and the ensemble classifier, respectively.

Embodiment 24. An ensemble model targeting a multiplicity of different inputs to provide a single unified classification, the model comprising:
a first fusion network trained on an optic nerve head (ONH) image dataset, the first fusion network comprising a first feature extraction module, a first feature fusion module, a first feature reconstruction module, and a first classifier;
a second fusion network trained on macular image dataset, the second fusion network comprising a second feature extraction module, a second feature fusion module, a second feature reconstruction module, and a second classifier; and
an ensemble model comprising the first fusion network, the second fusion network, an ensemble feature layer connected to an output of the first fusion network and to an output of the second fusion network, an ensemble classifier, and a combined classifier;
wherein the ensemble feature layer comprises a channel-level summation of a multiplicity of features extracted from the first fusion network and a multiplicity of features extracted from the second fusion network;
wherein the combined classifier is configured to return a binary classification indicating either AD-dementia or No-dementia through a method taking at least one input, respectively, from each of the first classifier, the second classifier, and the ensemble classifier.

Embodiment 25. The model according to Embodiment 24, wherein the first fusion network, the second fusion network, and the ensemble feature layer each, respectively, comprises a respective domain-specific batch normalization that comprises a respective first branch configured to accept feature extraction from a respective source domain, and a respective second branch configured to accept feature extraction from a respective target domain.

Embodiments of the subject invention address the technical problem of classifying a patient as dementia or no dementia being expensive, needing excessive imaging and data collection, and not being suitable for broad public health use and screening. This problem is addressed by providing digital image processing with a new ensemble based on optic nerve head-centered and macula-centered imaging, in which a machine learning method applying a combination of advanced techniques is utilized to provide a binary classification serving as part of a patient's identification for dementia or no dementia, useful in treatment of a patient for dementia or related diseases.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting of” or “consists essentially of” indicate that the claim encompasses embodiments containing the specified materials or steps and those that do not materially affect the basic and novel characteristic(s) of the claim. Use of the term “comprising” contemplates other embodiments that “consist of” or “consisting essentially of” the recited component(s).

When ranges are used herein, such as for dose ranges, combinations, and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e., the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

A greater understanding of the embodiments of the subject invention and of their many advantages may be had from the above examples, given by way of illustration. The examples are illustrative of some of the methods, applications, embodiments, and variants of the present invention. They are, of course, not to be considered as limiting the invention. Numerous changes and modifications can be made with respect to embodiments of the invention. It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Cheung, C. Y., et al. (2022). “A deep learning model for detection of Alzheimer's disease based on retinal photographs: a retrospective, multicentre case-control study.” Lancet Digital Health 4(11): e806-e815.
He, K., et al. (2015). “Deep Residual Learning for Image Recognition.” arXiv: 1512.03385v1.
Marshall, C. R. and I. Uchegbu “Artificial intelligence for detection of Alzheimer's disease: demonstration of real-world value is required to bridge the translational gap.” The Lancet Digital Health.
Nair, V. and G. E. Hinton (2010). Rectified linear units improve restricted boltzmann machines. Proceedings of the 27th international conference on machine learning (ICML-10).
Simonyan, K. and A. Zisserman (2015). “Very Deep Convolutional Networks for Large-Scale Image Recognition.” arXiv: 1409.1556v6.
Tian, J., et al. (2021). “Modular machine learning for Alzheimer's disease classification from retinal vasculature.” Scientific Reports 11(1): 238.
Wisely, C. E., et al. (2022). “Convolutional neural network to identify symptomatic Alzheimer's disease using multimodal retinal imaging.” British Journal of Ophthalmology 106(3): 388.

TABLE 1
Data distribution for the model training, internal validation, and external testing with different input data.

| | | Training | | | Internal validation | | | External testing-1 | | | External testing-2 | | |
|---|---|---|---|---|---|---|---|---|---|---|---|---|---|
| OCT | No. of | | AD- | No | | AD- | No | | AD- | No | | AD- | No |
| input | input | SUM | dementis | dementia | SUM | dementia | dementis | SUM | dementia | dementia | SUM | dementia | dementia |
| RNFL | Patient- | 802 | 188 | 614 | 50 | 20 | 30 | 38 | 12 | 26 | 60 | 34 | 26 |
| | level | | | | | | | | | | | | |
| | Scan- | 3039 | 1239 | 1800 | 189 | 113 | 76 | 86 | 27 | 59 | 125 | 72 | 53 |
| | level | | | | | | | | | | | | |
| GCIPL | Patient- | 802 | 188 | 614 | 50 | 20 | 30 | 38 | 12 | 26 | 60 | 34 | 26 |
| and MT | level | | | | | | | | | | | | |
| | Scan- | 3039 | 1239 | 1800 | 189 | 113 | 76 | 86 | 27 | 59 | 125 | 72 | 53 |
| | level | | | | | | | | | | | | |
| GCIPL, | Patient- | 802 | 188 | 614 | 50 | 20 | 30 | 38 | 12 | 26 | 60 | 34 | 26 |
| MT, and | level | | | | | | | | | | | | |
| RNFL | Scan- | 3039 | 1239 | 1800 | 189 | 113 | 76 | 86 | 27 | 59 | 125 | 72 | 53 |
| | level | | | | | | | | | | | | |

TABLE 2
The performance of the Fusion Network for Alzheimer's disease classification in the internal validation and external testing datasets.

| Model | Dataset | AUC | Accuracy, % | Sensitivity, % | Specificity, % | PPV, % | NPV, % |
|---|---|---|---|---|---|---|---|
| ONH | Internal | 0.898 (0.846-0.949) | 85.4 (80.1-90.7) | 90.5 (72.6-97.6) | 82.1 (70.2-95.5) | 86.4 (79.6-95.6) | 86.3 (72.6-96.1) |
| Model | validation | | | | | | |
| | External-1 | 0.767 (0.658-0.876) | 76.3 (56.3-85.0) | 73.1 (50.0-100) | 79.6 (35.2-94.4) | 62.1 (42.6-84.0) | 85.4 (78.0-100) |
| | External-2 | 0.664 (0.563-0.764) | 64.9 (56.8-73.9) | 50.8 (27.0-79.4) | 85.4 (54.2-100) | 81.8 (67.2-100) | 56.8 (50.0-69.6) |
| Macula | Internal | 0.875 (0.821-0.928) | 82.4 (76.5-87.6) | 78.8 (68.2-90.6) | 86.8 (72.1-95.6) | 88.2 (79.3-95.4) | 77.1 (69.0-87.5) |
| Model | validation | | | | | | |
| | External-1 | 0.787 (0.682-0.892) | 76.5 (60.5-85.2) | 76.9 (50.0-100) | 76.4 (43.6-96.4) | 60.7 (44.6-87.0) | 87.2 (78.6-100) |
| | External-2 | 0.737 (0.646-0.828) | 70.7 (62.9-78.5) | 72.7 (45.5-95.5) | 70.0 (40.0-92.0) | 76.0 (66.7-89.5) | 66.0 (54.6-88.2) |
| General | Internal | 0.917 (0.879-0.955) | 84.8 (79.2-89.9) | 80.0 (69.5-93.3) | 91.8 (75.3-97.3) | 92.9 (83.9-97.7) | 76.5 (68.3-88.9) |
| Model | validation | | | | | | |
| | External-1 | 0.770 (0.663-0.877) | 74.1 (61.7-85.2) | 80.0 (56.0-100) | 73.2 (48.2-91.1) | 56.3 (43.6-76.9) | 89.6 (81.4-100) |
| | External-2 | 0.740 (0.649-0.832) | 71.7 (64.1-79.2) | 75.4 (50.7-95.7) | 70.6 (41.2-90.2) | 77.0 (67.7-89.1) | 66.7 (55.4-88.9) |
| Ensemble | Internal | 0.943 (0.906-0.980) | 90.5 (86.0-94.4) | 93.3 (84.8-99.1) | 87.7 (76.7-95.9) | 91.5 (86.0-96.8) | 90.0 (80.5-98.4) |
| Model | validation | | | | | | |
| | External-1 | 0.786 (0.673-0.899) | 80.3 (63.0-87.7) | 68.0 (44.0-96.0) | 87.5 (51.8-100) | 69.6 (44.9-100) | 85.3 (78.6-96.4) |
| | External-2 | 0.795 (0.716-0.874) | 74.2 (66.7-81.7) | 68.1 (50.7-88.4) | 84.3 (60.8-96.1) | 85.3 (73.7-95.2) | 65.8 (57.1-81.3) |

We claim:

1. A system for operating an ensemble model targeting a multiplicity of inputs to provide a single unified classification, the system comprising:
a first fusion network trained on an optic nerve head (ONH) image dataset;
a second fusion network trained on a macular image dataset; and
an ensemble feature layer comprising a channel-level summation of a plurality of features extracted from the first fusion network and a plurality of features extracted from the second fusion network.

2. The system according to claim 1, wherein:
the first fusion network comprises a first local classifier;
the second fusion network comprises a second local classifier; and
the system further comprises an ensemble classifier.

3. The system according to claim 2, wherein the first local classifier comprises a first plurality of features from the first fusion network; and the second local classifier comprises a second plurality of features from the second fusion network.

4. The system according to claim 3, wherein the ensemble classifier comprises a third plurality of aggregated features comprising at least one feature from the first plurality of features and at least one feature from the second plurality of features.

5. The system according to claim 4, wherein the ensemble classifier is configured to return a binary classification indicating either AD-dementia or No-dementia through a majority voting method taking at least one input, respectively, from each of the first plurality of features, the second plurality of features, and the third plurality of aggregated features.

6. The system according to claim 1, wherein:
the first fusion network comprises a first feature extraction component, a first feature fusion component, and a first feature reconstruction component; and
the second fusion network comprises a second feature extraction component, a second feature fusion component, and a second feature reconstruction component.

7. The system according to claim 6, wherein:
the first feature extraction component comprises a first plurality of feature maps, each respective feature map in the first plurality of feature maps having a unique convolution kernel weighting; and
the second feature extraction component comprises a second plurality of feature maps, each respective feature map in the second plurality of feature maps having a unique convolution kernel weighting.

8. The system according to claim 6, wherein:
the first feature fusion component comprises a first intermediate node that integrates extracted image features from a first plurality of feature extraction channels; and
the second feature fusion component comprises a second intermediate node that integrates extracted image features from a second plurality of feature extraction channels.

9. The system according to claim 6, wherein:
the first feature extraction component comprises a first plurality of residual blocks; and
the second feature extraction component comprises a second plurality of residual blocks.

10. The system according to claim 6, wherein:
the first fusion network is based on a first inter-domain correlation between a first source domain comprising a first training data set and a first target domain comprising a first external test data set; and
the second fusion network is based on a second inter-domain correlation between a second source domain comprising a second training data set and a second target domain comprising a second external test data set.

11. The system according to claim 10, wherein the first fusion network, the second fusion network, and the ensemble feature layer each, respectively, comprises a respective domain specific batch normalization module.

12. The system according to claim 11, wherein each respective domain specific batch normalization module comprises a first branch component configured to accept feature extraction from a respective source domain, and a second branch component configured to accept feature extraction from a respective target domain.

13. A method for generating a binary classification of Alzheimer's Disease (AD) in a patient as either (a) AD-dementia or (b) no dementia, the method comprising:
creating a deep learning system by performing following steps:
a) providing a first fusion network comprising a first feature extraction module, a first feature fusion module, a first feature reconstruction module, and a first classifier;
b) training the first fusion network on a first image set comprising a plurality of optical coherence tomography (OCT) optic nerve head (ONH) image channels;
c) providing a second fusion network comprising a second feature extraction module, a second feature fusion module, a second feature reconstruction module, and a second classifier;
d) training the second fusion network on a second image set comprising a plurality of optical coherence tomography (OCT) macula-centered image channels;
e) providing an ensemble model comprising the first fusion network, the second fusion network, an ensemble feature layer connected to an output of the first fusion network and to an output of the second fusion network, and an ensemble classifier;
f) training the ensemble model on the combined first image set and second image set;
obtaining a patient-specific OCT image set; and
processing the patient-specific OCT image set through the ensemble model, and generating the binary classification of Alzheimer's disease in the patient as either (a) AD-dementia or (b) no dementia.

14. The method according to claim 13, wherein the plurality of OCT ONH image channels comprises at least one of retinal nerve fiber layer (RNFL) thickness map images, RNFL deviation map images, and ONH-centered en face images, each respective image channel comprising single eye data.

15. The method according to claim 13, wherein the plurality of OCT ONH image channels comprise retinal nerve fiber layer (RNFL) thickness map images, RNFL deviation map images, and ONH-centered en face images, each respective image channel comprising single eye data.

16. The method according to claim 13, wherein the plurality of OCT macula-centered image channels comprises at least one of ganglion cell inner plexiform layer (GCIPL) thickness map images, GCIPL deviation map images, macular thickness map images, and macula-centered en face images, each respective image channel comprising single eye data.

17. The method according to claim 13, wherein the plurality of OCT ONH image channels comprise ganglion cell inner plexiform layer (GCIPL) thickness map images, GCIPL deviation map images, macular thickness map images, and macula-centered en face images, each respective image channel comprising single eye data.

18. The method according to claim 13, wherein the provided ensemble model is configured to generate the final classification results through majority voting including outputs from the first classifier, the second classifier, and the ensemble classifier, respectively.

19. An ensemble model targeting a plurality of different inputs to provide a single unified classification, the model comprising:
a first fusion network trained on an optic nerve head (ONH) image dataset, the first fusion network comprising a first feature extraction module, a first feature fusion module, a first feature reconstruction module, and a first classifier;
a second fusion network trained on macular image dataset, the second fusion network comprising a second feature extraction module, a second feature fusion module, a second feature reconstruction module, and a second classifier; and
an ensemble model comprising the first fusion network, the second fusion network, an ensemble feature layer connected to an output of the first fusion network and to an output of the second fusion network, an ensemble classifier and a combined classifier;
wherein the ensemble feature layer comprises a channel level summation of a plurality of features extracted from the first fusion network and a plurality of features extracted from the second fusion network;
wherein the combined classifier is configured to return a binary classification indicating either AD-dementia or No-dementia through a method taking at least one input, respectively, from each of the first classifier, the second classifier, and the ensemble classifier.

20. The model according to claim 19, wherein the first fusion network, the second fusion network, and the ensemble feature layer each, respectively, comprises a respective domain specific batch normalization that comprises a respective first branch configured to accept feature extraction from a respective source domain, and a respective second branch configured to accept feature extraction from a respective target domain.
